(19) United States
(12) Patent Application Publication
(10) Pub. No.: US 2017/0121690 A1
(43) Pub. Date: May 4, 2017

(54) LACCASE VARIANTS WITH IMPROVED PROPERTIES

(71) Applicant: METGEN OY, Kaarina (FI)

(72) Inventor: Klara Birikh, Kaarina (FI)

(21) Appl. No.: 15/128,916

(22) PCT Filed: Mar. 24, 2015
(86) PCT No.: PCT/EP2015/056211

§ 371 Date: Sep. 23, 2016

(30) Foreign Application Priority Data
Mar. 24, 2014 (EP) 14161322.4
Apr. 8, 2014 (EP) 14163949.2

(51) Int. Cl. C12N 9/02

(52) U.S. Cl. CPC C12N 9/0061; C12Y 110/03002

(57) ABSTRACT

The present application relates to laccase variants and uses thereof as eco-friendly biocatalysts in various industrial processes. More in particular, the application relates to a polypeptide with laccase activity comprising an amino acid sequence that is at least 60% identical to the amino acid sequence according to SEQ ID NO: 1, wherein the polypeptide comprises an alanine residue at a position corresponding to amino acid 260 of SEQ ID NO: 1.

### CROSS-REFERENCE TO RELATED APPLICATIONS

This application is a national phase entry under 35 U.S.C. §371 of International Patent Application PCT/EP2015/056211, filed Mar. 24, 2015, designating the United States of America and published in English as International Patent Publication WO 2015/144679 A1 on Oct. 1, 2015, which claims the benefit under Article 8 of the Patent Cooperation Treaty to European Patent Application Serial No. 14163949.2, filed Apr. 8, 2014, and to European Patent Application Serial No. 14161322.4, filed Mar. 24, 2014.

### STATEMENT ACCORDING TO 37 C.F.R. §1.821(C) OR (E)—SEQUENCE LISTING SUBMITTED AS ASCII TEXT FILE

Pursuant to 37 C.F.R. §1.821(c) or (e), a file containing an ASCII text version of the Sequence Listing has been submitted concomitant with this application, the contents of which are hereby incorporated by reference.

### TECHNICAL FIELD

The present application relates to laccase variants and uses thereof as eco-friendly biocatalysts in various industrial processes.

### BACKGROUND

Laccases (EC 1.10.3.2) are enzymes having a wide taxonomic distribution and belonging to the group of multicopper oxidases. Laccases are eco-friendly catalysts, which use molecular oxygen from air to oxidize various phenolic and non-phenolic lignin-related compounds as well as highly recalcitrant environmental pollutants, and produce water as the only side product. These natural “green” catalysts are used for diverse industrial applications including the detoxification of industrial effluents, mostly from the paper and pulp, textile and petrochemical industries, and used as bioremediation agent to clean up herbicides, pesticides and certain explosives in soil. Laccases are also used as cleaning agents for certain water purification systems. In addition, their capacity to remove xenobiotic substances and produce polymeric products makes them a useful tool for bioremediation purposes. Another large proposed application area of laccases is biomass pretreatment in biofuel and in the pulp and paper industry.

Laccase molecules are usually monomers consisting of three consecutively connected cupredoxin-like domains twisted in a tight globule. The active site of laccases contains four copper ions: a mononuclear “blue” copper ion (T1 site) and a three-nuclear copper cluster (T2/T3 site) consisting of one T2 copper ion and two T3 copper ions.

Laccases may be isolated from different sources such as plants, fungi or bacteria and are very diverse in primary sequences. However, they have some conserved regions in the sequences and certain common features in their three-dimensional structures. A comparison of sequences of more than 100 laccases has revealed four short conservative regions (no longer than 10 aa each) that are specific for all laccases.^{(7, 8)} One cysteine and ten histidine residues form a ligand environment of copper ions of the laccase active site present in these four conservative amino acid sequences.

The best studied bacterial laccase is CotA laccase. CotA is a component of the outer coat layers of bacillus endospore. It is a 65-kDa protein encoded by the CotA gene.⁽¹⁾

CotA belongs to a diverse group of multi-copper “blue” oxidases that includes the laccases. This protein demonstrates high thermostability, and resistance to various hazardous elements in accordance with the survival abilities of the endospore.

Recombinant protein expression in easily cultivatable hosts can allow higher productivity in shorter time and reduces the costs of production. The versatility and scaling-up possibilities of the recombinant protein production opened up new commercial opportunities for their industrial uses. Moreover, protein production from pathogenic or toxin-producing species can take advantage of safer or even GRAS (generally recognized as safe) microbial hosts. In addition, protein engineering can be employed to improve the stability, activity and/or specificity of an enzyme, thus tailor-made enzymes can be produced to suit the requirement of the users or of the process.

Enzyme productivity can be increased by the use of multiple gene copies, strong promoters and efficient signal sequences, properly designed to address proteins to the extracellular medium, thus simplifying downstream processing.

Recombinant protein yield in bacterial hosts is often limited by the inability of the protein to fold into correct 3D-structure upon biosynthesis of the polypeptide chain. This may cause exposure of hydrophobic patches on the surface of the protein globule and result in protein aggregation. Mechanisms of heterologous protein folding in vivo are poorly understood, and foldability of different proteins in bacteria is unpredictable.

Yield of soluble active protein can be sometimes improved by changing cultivation conditions. In addition, there are examples when protein yield was improved by introducing single point mutations in the protein sequence. However, no rationale has been identified behind finding suitable mutations.

Heterologous expression of laccase in Escherichia coli has often been used as a strategy to get around the problem of obtaining laccases that are not easily producible in natural hosts. The recombinant expression of Bacillus subtilis CotA in E. coli has allowed its deep characterization, structure solving, and functional evolution.^{(1, 2, 3)} However, very often the production yield is low, due to a strong tendency of this enzyme to form aggregates that render the protein irreversibly inactive.⁽⁴⁾ This tendency has been attributed to the fact that, in nature, CotA laccase is integrated in a spore coat structure via interaction with other protein components, and it is likely that correct laccase folding is enhanced by interaction with other proteins. When this laccase is recombinantly expressed as an individual polypeptide, those supporting interactions are missing and many miss-folded proteins form aggregates in bacterial cells. When expressed in higher microorganisms such as yeast, for a large part, misfolded laccase molecules are degraded.

There is a need in the art for means and methods for improving the yield of laccases in heterologous expression systems. This is particularly true for bacterial laccases, such as CotA laccases.

### BRIEF SUMMARY

This disclosure addresses this need in that it provides variant laccases with improved properties. More in particular, the disclosure relates to a polypeptide with laccase activity comprising an amino acid sequence that is at least 60% identical to the amino acid sequence according to SEQ ID NO: 1, wherein the polypeptide comprises an alanine residue at a position corresponding to amino acid 260 of SEQ ID NO: 1.

In addition, the disclosure provides improved nucleic acids, vectors and compositions encoding the variant laccase enzymes according to the disclosure.

The disclosure also provides recombinant heterologous expression systems such as host cells comprising a nucleic acid, a vector or a composition according to the disclosure.

Also provided herein are methods for producing a polypeptide according to the disclosure, comprising the steps of:
a. culturing a recombinant host cell comprising a polynucleotide according to the disclosure under conditions suitable for the production of the polypeptide, and
b. recovering the polypeptide obtained, and
c. optionally purifying the polypeptide.

The disclosure also relates to the use of a polypeptide according to the disclosure in an application selected from the group consisting of pulp delignification, degrading or decreasing the structural integrity of lignocellulosic material, textile dye bleaching, wastewater detoxification, xenobiotic detoxification, production of a sugar from a lignocellulosic material and recovering cellulose from a biomass.

The disclosure also relates to a method for improving the yield of a polypeptide with laccase activity in a heterologous expression system comprising the step of altering the amino acid of that polypeptide at a position corresponding to position 260 in SEQ ID NO: 1 to an alanine residue.

### BRIEF DESCRIPTION OF THE DRAWINGS

FIG. 1: Relative increase of volumetric activity. Graph showing the relative increase of volumetric activity in parallel cultures in E. coli of wild-type (non-mutated) versus mutated laccases. The abbreviation “SEQ” followed by a number refers to the SEQ ID NO: of the respective number; “SEQ1” refers to SEQ ID NO: 1. “SEQ 1 260A” refers to the polypeptide according to SEQ ID NO: 1 wherein the amino acid corresponding to position 260 is replaced by an A (Ala or alanine).

FIG. 2: Relative increase of volumetric activity. Graph showing the relative increase of volumetric activity in parallel cultures in Pichia pastoris of wild-type (non-mutated) versus mutated laccases. The abbreviation “SEQ” followed by a number refers to the SEQ ID NO: of the respective number; “SEQ1” refers to SEQ ID NO: 1. “SEQ 1 260A” refers to the polypeptide according to SEQ ID NO: 1 wherein the amino acid corresponding to position 260 is replaced by an Alanine residue (Ala or A).

### DETAILED DESCRIPTION

This disclosure is based on the observation that a single amino acid substitution in different laccases improves the yield of that laccase by at least 50% when expressed in prokaryotes as well as in eukaryotes. It was also found that the variant laccase remains active.

The term “amino acid substitution” is used herein the same way as it is commonly used, i.e., the term refers to a replacement of one or more amino acids in a protein with another. Artificial amino acid substitutions may also be referred to as mutations.

SEQ ID NO: 1 is a CotA laccase from Bacillus subtilis newly disclosed herein, whereas SEQ ID NO: 2 is a CotA laccase that has been previously disclosed in WO 2013/038062. It was found that laccase variants that have an alanine residue at an amino acid position corresponding to position 260 (260A1a) in SEQ ID NO: 1 provided a higher yield when expressed in a heterologous expression system.

SEQ ID NO: 3 and SEQ ID NO: 4 disclose B. subtilis spore coat proteins with laccase activity (CotA laccase) that carry such a mutation. In fact, SEQ ID NO: 3 is a variant from SEQ ID NO: 1 wherein a threonine residue at position 260 has been replaced by an alanine residue. SEQ ID NO: 4 is a variant from SEQ ID NO: 2 wherein a threonine residue at position 260 has been replaced by an alanine residue.

A homology search was performed for proteins homologous to SEQ ID NO: 1 using SEQ ID NO: 1 as the query sequence in the “Standard protein BLAST” software, available at http://blast.ncbi.nlm.nih.gov/Blast.cgi?PROGRAM=blastp&PAGE_TYPE=BlastSearch& LINK_LOC=blasthome. More information on the software and database versions is available at the National Center for Biotechnology Information at National library of Medicine at National Institute of Health internet site at ncbi.nlm.nih.gov. Therein, a number of molecular biology tools including BLAST (Basic Logical Alignment Search Tool) is to be found. BLAST makes use of the following databases: all non-redundant GenBank CDS translations+PDB+SwissProt+PIR+PRF excluding environmental samples from WGS projects. The search as reported herein was performed online on 19 Feb. 2014 and employed BLASTP version 2.2.29+.

The search revealed 69 sequences with at least 60% sequence identity to SEQ ID NO: 1 (Table 1).

TABLE 1
Sequences obtained from a BLAST search disclosing 69 sequences with at least 60% identity to SEQ ID NO: 1.

| | | | | | AA # | AA at pos |
|---|---|---|---|---|---|---|
| SEQ | BLAST | | | Overall | corr. to | corr. to |
| ID NO: | No: | Description | Accession No: | identity⁽¹⁾ | pos 260⁽²⁾ | AA⁽³⁾ |
| 1 | 1 | CotA laccase from B. subtilis (query sequence) | | 100% | 260 | T |
| 25 | 2 | laccase [Bacillus subtilis] | AGZ16504.1 | 98% | 260 | T |
| 26 | 3 | spore copper-dependent laccase (outer coat) [Bacillus | YP_003865004.1 | 98% | 260 | T |
| | | subtilis subsp. spizizenii str. W23] | | | | |
| | | >ref\|WP_003219376.1\|copper oxidase [Bacillus | | | | |
| | | subtilis] >gb\|EFG93543.1\|spore copper-dependent | | | | |
| | | laccase [Bacillus subtilis subsp. spizizenii ATCC 6633] | | | | |
| | | >gb\|ADM36695.1\|spore copper-dependent laccase | | | | |
| | | (outer coat) [Bacillus subtilis subsp. spizizenii str. | | | | |
| | | W23] | | | | |
| 27 | 4 | spore copper-dependent laccase [Bacillus subtilis] | WP_004397739.1 | 96% | 260 | T |
| | | >gb\|ELS60660.1\|spore copper-dependent laccase | | | | |
| | | [Bacillus subtilis subsp. inaquosorum KCTC 13429] | | | | |
| 28 | 5 | copper oxidase [Bacillus subtilis] | WP_019713492.1 | 96% | 260 | T |
| 29 | 6 | laccase [Bacillus vallismortis] | AGR50961.1 | 95% | 260 | T |
| 30 | 7 | spore coat protein A [Bacillus subtilis XF-1] | YP_007425830.1 | 96% | 262 | T |
| | | >ref\|WP_015382982.1\|spore coat protein A [Bacillus] | | | | |
| | | >gb\|AGE62493.1\|spore coat protein A [Bacillus | | | | |
| | | subtilis XF-1] >gb\|ERI42893.1\|copper oxidase | | | | |
| | | [Bacillus sp. EGD-AK10] | | | | |
| 31 | 8 | spore copper-dependent laccase [Bacillus subtilis | YP_004206641.1 | 96% | 260 | T |
| | | BSn5] >ref\|YP_005559844.1\|spore coat protein A | | | | |
| | | [Bacillus subtilis subsp. natto BEST195] | | | | |
| | | >ref\|YP_007210655.1\|Spore coat protein A [Bacillus | | | | |
| | | subtilis subsp. subtilis str. BSP1] | | | | |
| | | >ref\|WP_014479048.1\|copper oxidase [Bacillus | | | | |
| | | subtilis] >dbj\|BAI84141.1\|spore coat protein A | | | | |
| | | [Bacillus subtilis subsp. natto BEST195] | | | | |
| | | >gb\|ADV95614.1\|spore copper-dependent laccase | | | | |
| | | [Bacillus subtilis BSn5] >gb\|ADZ57279.1\|laccase | | | | |
| | | [Bacillus sp. LS02] >gb\|ADZ57280.1\|laccase [Bacillus | | | | |
| | | sp. LS03] >gb\|ADZ57283.1\|laccase [Bacillus sp. | | | | |
| | | WN01] >gb\|ADZ57284.1\|laccase [Bacillus subtilis] | | | | |
| | | >gb\|AGA20638.1\|Spore coat protein A [Bacillus | | | | |
| | | subtilis subsp. subtilis str. BSP1] | | | | |
| 32 | 9 | CotA [Bacillus sp. JS] >ref\|WP_014663045.1\|copper | YP_006230497.1 | 95% | 260 | T |
| | | oxidase [Bacillus sp. JS] >gb\|AFI27241.1\|CotA | | | | |
| | | [Bacillus sp. JS] | | | | |
| 33 | 10 | copper oxidase [Bacillus subtilis QH-1] | EXF51833.1 | 95% | 260 | T |
| 34 | 11 | copper oxidase [Bacillus subtilis] >gb\|EHA29133.1\| | WP_003234000.1 | 95% | 262 | T |
| | | spore copper-dependent laccase [Bacillus subtilis | | | | |
| | | subsp. subtilis str. SC-8] | | | | |
| 35 | 12 | outer spore coat copper-dependent laccase [Bacillus | YP_006628799.1 | 95% | 262 | T |
| | | subtilis QB928] >ref\|WP_014906195.1\|copper oxidase | | | | |
| | | [Bacillus subtilis] >dbj\|BAA22774.1\|spore coat proein | | | | |
| | | A [Bacillus subtilis] >gb\|AFQ56549.1\|Outer spore | | | | |
| | | coat copper-dependent laccase [Bacillus subtilis | | | | |
| | | QB928] | | | | |
| 36 | 13 | spore coat protein A [Bacillus subtilis subsp. subtilis | NP_388511.1 | 95% | 260 | T |
| | | str. 168] | | | | |
| 37 | 14 | spore coat protein A [Bacillus subtilis subsp. subtilis | YP_007661398.1 | 95% | 260 | T |
| | | str. BAB-1] >ref\|WP_015482891.1\|spore coat protein | | | | |
| | | A [Bacillus subtilis] >gb\|AGI27890.1\|spore coat | | | | |
| | | protein A [Bacillus subtilis subsp. subtilis str. BAB-1] | | | | |
| 38 | 15 | Chain A, Mutations In The Neighbourhood of CotA- | 4AKQ_A | 95% | 260 | T |
| | | Laccase Trinuclear Site: E498d Mutant | | | | |
| 39 | 16 | Chain A, Mutations In The Neighbourhood of CotA- | 4A68_A | 95% | 260 | T |
| | | Laccase Trinuclear Site: D116n Mutant | | | | |
| 40 | 17 | Chain A, Mutations In The Neighbourhood of CotA- | 4A66_A | 95% | 260 | T |
| | | Laccase Trinuclear Site: D116a Mutant | | | | |
| 41 | 18 | spore coat protein [Bacillus subtilis] | ACS44284.1 | 95% | 260 | T |
| 42 | 19 | spore coat protein [Bacillus subtilis] | AGK12417.1 | 95% | 260 | T |
| 43 | 20 | Chain A, Crystal Structure Of The Reconstituted CotA | 2X87_A | 95% | 260 | T |
| 44 | 21 | laccase [Bacillus sp. ZW2531-1] | AFN66123.1 | 95% | 260 | T |
| 45 | 22 | Chain A, Mutations In The Neighbourhood of CotA- | 4A67_A | 95% | 260 | T |
| | | Laccase Trinuclear Site: D116e Mutant | | | | |
| 46 | 23 | Chain A, Proximal Mutations At The Type 1 Cu Site of | 2WSD_A | 95% | 260 | T |
| | | CotA-Laccase: I494a Mutant | | | | |
| 47 | 24 | Chain A, Mutations In The Neighbourhood of CotA- | 4AKP_A | 95% | 260 | T |
| | | Laccase Trinuclear Site: e498t Mutant | | | | |
| 48 | 25 | laccase [Bacillus sp. HR03] | ACM46021.1 | 94% | 260 | T |
| 49 | 26 | copper oxidase [Bacillus vallismortis] | WP_010329056.1 | 94% | 260 | T |
| 50 | 27 | laccase [Bacillus subtilis] | AEK80414.1 | 92% | 260 | T |
| 51 | 28 | copper oxidase [Bacillus mojavensis] | WP_010333230.1 | 91% | 260 | T |
| 52 | 29 | Chain A, Mutations In The Neighbourhood of CotA- | 4AKO_A | 94% | 260 | T |
| | | Laccase Trinuclear Site: E4981 Mutant | | | | |
| 53 | 30 | CotA [Bacillus subtilis] | AAB62305.1 | 89% | 260 | T |
| 54 | 31 | spore copper-dependent laccase [Bacillus atrophaeus | YP_003972023.1 | 81% | 260 | T |
| | | 1942] >ref\|WP_003328493.1\|copper oxidase [Bacillus | | | | |
| | | atrophaeus] >gb\|ADP31092.1\|spore copper-dependent | | | | |
| | | laccase (outer coat) [Bacillus atrophaeus 1942] | | | | |
| | | >gb\|EIM09308.1\|spore copper-dependent laccase | | | | |
| | | [Bacillus atrophaeus C89] | | | | |
| 55 | 32 | Spore coat protein A [Bacillus atrophaeus] | WP_010787813.1 | 81% | 260 | T |
| | | >gb\|EOB38473.1\|Spore coat protein A [Bacillus | | | | |
| | | atrophaeus UCMB-5137] | | | | |
| 56 | 33 | copper oxidase [Bacillus sp. 5B6] >gb\|EIF12180.1\| | WP_007609818.1 | 77% | 260 | T |
| | | CotA [Bacillus sp. 5B6] | | | | |
| 57 | 34 | outer spore coat copper-dependent laccase [Bacillus | YP_007496315.1 | 77% | 260 | T |
| | | amyloliquefaciens subsp. plantarum UCMB5036] | | | | |
| | | >ref\|YP_008411651.1\|outer spore coat copper- | | | | |
| | | dependent laccase [Bacillus amyloliquefaciens subsp. | | | | |
| | | plantarum UCMB5033] >ref\|YP_008420054.1\|outer | | | | |
| | | spore coat copper-dependent laccase [Bacillus | | | | |
| | | amyloliquefaciens subsp. plantarum UCMB5113] | | | | |
| | | >ref\|WP_015416957.1\|outer spore coat copper- | | | | |
| | | dependent laccase [Bacillus amyloliquefaciens] | | | | |
| | | >emb\|CCP20645.1\|outer spore coat copper-dependent | | | | |
| | | laccase [Bacillus amyloliquefaciens subsp. plantarum | | | | |
| | | UCMB5036] >emb\|CDG28620.1\|outer spore coat | | | | |
| | | copper-dependent laccase [Bacillus amyloliquefaciens | | | | |
| | | subsp. plantarum UCMB5033] >emb\|CDG24919.1\| | | | | |
| | | outer spore coat copper-dependent laccase [Bacillus | | | | |
| | | amyloliquefaciens subsp. plantarum UCMB5113] | | | | |
| 58 | 35 | spore coat protein CotA [Bacillus amyloliquefaciens | YP_005419918.1 | 77% | 260 | T |
| | | subsp. plantarum YAU B9601-Y2] | | | | |
| | | >ref\|YP_006327430.1\|spore coat protein A [Bacillus | | | | |
| | | amyloliquefaciens Y2] >ref\|WP_014417082.1\|copper | | | | |
| | | oxidase [Bacillus amyloliquefaciens] | | | | |
| | | >gb\|ADZ57285.1\|laccase [Bacillus sp. LC02] | | | | |
| | | >emb\|CCG48602.1\|spore coat protein CotA [Bacillus | | | | |
| | | amyloliquefaciens subsp. plantarum YAU B9601-Y2] | | | | |
| | | >gb\|AFJ60705.1\|spore coat protein A [Bacillus | | | | |
| | | amyloliquefaciens Y2] >dbj\|BAM49543.1\|spore | | | | |
| | | copper-dependent laccase [Bacillus subtilis BEST7613] | | | | |
| | | >dbj\|BAM56813.1\|spore copper-dependent laccase | | | | |
| | | [Bacillus subtilis BEST7003] | | | | |
| 59 | 36 | bilirubin oxidase [Bacillus amyloliquefaciens subsp. | YP_008625231.1 | 77% | 260 | T |
| | | plantarum NAU-B3] >ref\|WP_022552695.1\|bilirubin | | | | |
| | | oxidase [Bacillus amyloliquefaciens] | | | | |
| | | >emb\|CDH94370.1\|bilirubin oxidase [Bacillus | | | | |
| | | amyloliquefaciens subsp. plantarum NAU-B3] | | | | |
| 60 | 37 | spore coat protein A [Bacillus amyloliquefaciens | YP_007185316.1 | 77% | 260 | T |
| | | subsp. plantarum AS43.3] >ref\|WP_015239305.1\| | | | | |
| | | spore coat protein A [Bacillus amyloliquefaciens] | | | | |
| | | >gb\|AFZ89646.1\|spore coat protein A [Bacillus | | | | |
| | | amyloliquefaciens subsp. plantarum AS43.3] | | | | |
| 61 | 38 | CotA [Bacillus amyloliquefaciens subsp. plantarum str. | YP_001420286.1 | 77% | 260 | T |
| | | FZB42] >ref\|YP_008725930.1\|CotA [Bacillus | | | | |
| | | amyloliquefaciens CC178] >ref\|WP_012116986.1\| | | | | |
| | | copper oxidase [Bacillus amyloliquefaciens] | | | | |
| | | >gb\|ABS73055.1\|CotA [Bacillus amyloliquefaciens | | | | |
| | | subsp. plantarum str. FZB42] >gb\|AGZ55352.1\|CotA | | | | |
| | | [Bacillus amyloliquefaciens CC178] | | | | |
| 62 | 39 | laccase [Bacillus sp. LC03] | ADZ57286.1 | 76% | 260 | T |
| 63 | 40 | copper oxidase [Bacillus sp. 916] >gb\|EJD67619.1\| | WP_007408880.1 | 77% | 260 | T |
| | | CotA [Bacillus sp. 916] | | | | |
| 64 | 41 | copper oxidase [Bacillus amyloliquefaciens] | WP_021495201.1 | 76% | 260 | T |
| | | >gb\|ERH51073.1\|copper oxidase [Bacillus | | | | |
| | | amyloliquefaciens EGD-AQ14] | | | | |
| 65 | 42 | bilirubin oxidase [Bacillus amyloliquefaciens subsp. | YP_005129370.1 | 76% | 260 | T |
| | | plantarum CAU B946] >ref\|YP_007446652.1\|bilirubin | | | | |
| | | oxidase [Bacillus amyloliquefaciens IT-45] | | | | |
| | | >ref\|YP_008949033.1\|copper oxidase [Bacillus | | | | |
| | | amyloliquefaciens LFB112] >ref\|WP_003155789.1\| | | | | |
| | | copper oxidase [Bacillus amyloliquefaciens] | | | | |
| | | >gb\|ADZ57278.1\|laccase [Bacillus sp. LS01] | | | | |
| | | >gb\|ADZ57282.1\|laccase [Bacillus sp. LS05] | | | | |
| | | >emb\|CCF04175.1\|bilirubin oxidase [Bacillus | | | | |
| | | amyloliquefaciens subsp. plantarum CAU B946] | | | | |
| | | >gb\|EKE46469.1\|bilirubin oxidase [Bacillus | | | | |
| | | amyloliquefaciens subsp. plantarum M27] | | | | |
| | | >gb\|AGF28771.1\|bilirubin oxidase [Bacillus | | | | |
| | | amyloliquefaciens IT-45] >gb\|ERK81509.1\|copper | | | | |
| | | oxidase [Bacillus amyloliquefaciens UASWS BA1] | | | | |
| | | >gb\|AHC41184.1\|copper oxidase [Bacillus | | | | |
| | | amyloliquefaciens LFB112] | | | | |
| 66 | 43 | copper oxidase [Bacillus amyloliquefaciens subsp. | AHK48246.1 | 76% | 260 | T |
| | | plantarum TrigoCor1448] | | | | |
| 67 and 5 | 44 | spore copper-dependent laccase [Bacillus | YP_003919218.1 | 76% | 260 | T |
| | | amyloliquefaciens DSM 7] >ref\|YP_005540261.1\| | | | | |
| | | spore copper-dependent laccase [Bacillus | | | | |
| | | amyloliquefaciens TA208] >ref\|YP_005544441.1\| | | | | |
| | | spore copper-dependent laccase [Bacillus | | | | |
| | | amyloliquefaciens LL3] >ref\|YP_005548603.1\|spore | | | | |
| | | copper-dependent laccase [Bacillus amyloliquefaciens | | | | |
| | | XH7] >ref\|WP_013351262.1\|copper oxidase [Bacillus | | | | |
| | | amyloliquefaciens] >emb\|CBI41748.1\|spore copper- | | | | |
| | | dependent laccase [Bacillus amyloliquefaciens DSM 7] | | | | |
| | | >gb\|AEB22768.1\|spore copper-dependent laccase | | | | |
| | | [Bacillus amyloliquefaciens TA208] >gb\|AEB62213.1\| | | | | |
| | | spore copper-dependent laccase [Bacillus | | | | |
| | | amyloliquefaciens LL3] >gb\|AEK87755.1\|spore | | | | |
| | | copper-dependent laccase [Bacillus amyloliquefaciens | | | | |
| | | XH7] | | | | |
| 68 and 6 | 45 | copper oxidase [Bacillus siamensis] | WP_016937040.1 | 75% | 260 | M |
| 69 | 46 | outer spore coat protein CotA [Bacillus sonorensis] | WP_006637314.1 | 67% | 258 | T |
| | | >gb\|EME75462.1\|outer spore coat protein CotA | | | | |
| | | [Bacillus sonorensis L12] | | | | |
| 70 | 47 | copper oxidase [Bacillus sp. M 2-6] >gb\|EIL85237.1\| | WP_008344352.1 | 67% | 260 | T |
| | | outer spore coat protein A [Bacillus sp. M 2-6] | | | | |
| 71 | 48 | spore copper-dependent laccase [Bacillus | WP_007496963.1 | 67% | 260 | T |
| | | stratosphericus] >gb\|EMI14845.1\|spore copper- | | | | |
| | | dependent laccase [Bacillus stratosphericus LAMA | | | | |
| | | 585] | | | | |
| 72 | 49 | copper oxidase [Bacillus pumilus] | WP_017359847.1 | 67% | 260 | T |
| 73 | 50 | CotA [Bacillus pumilus] | AEX93437.1 | 67% | 260 | T |
| 74 | 51 | copper oxidase [Bacillus pumilus] >gb\|EDW21710.1\| | WP_003213818.1 | 67% | 260 | T |
| | | spore coat protein A [Bacillus pumilus ATCC 7061] | | | | |
| 75 | 52 | CotA [Bacillus pumilus] | AFL56752.1 | 67% | 260 | T |
| 76 | 53 | copper oxidase [Bacillus pumilus] | WP_019743779.1 | 67% | 260 | T |
| 77 | 54 | CotA [Bacillus pumilus] | AFK33221.1 | 67% | 260 | T |
| 78 | 55 | outer spore coat protein A [Bacillus pumilus SAFR- | YP_001485796.1 | 67% | 260 | T |
| | | 032] >ref\|WP_012009087.1\|copper oxidase [Bacillus | | | | |
| | | pumilus] >gb\|ABV61236.1\|outer spore coat protein A | | | | |
| | | [Bacillus pumilus SAFR-032] | | | | |
| 79 | 56 | copper oxidase [Bacillus sp. HYC-10] | WP_008355710.1 | 66% | 260 | T |
| | | >gb\|EKF36812.1\|outer spore coat protein A [Bacillus | | | | |
| | | sp. HYC-10] | | | | |
| 80 | 57 | copper oxidase [Bacillus sp. CPSM8] | WP_023855578.1 | 65% | 258 | T |
| | | >gb\|ETB72519.1\|copper oxidase [Bacillus sp. | | | | |
| | | CPSM8] | | | | |
| 81 | 58 | outer spore coat protein CotA [Bacillus licheniformis | YP_008076901.1 | 65% | 258 | T |
| | | 9945A] >ref\|WP_020450420.1\|outer spore coat | | | | |
| | | protein CotA [Bacillus licheniformis] | | | | |
| | | >gb\|AGN35164.1\|outer spore coat protein CotA | | | | |
| | | [Bacillus licheniformis 9945A] | | | | |
| 82 | 59 | laccase [Bacillus sp. 2008-12-5] | AFP45763.1 | 67% | 261 | T |
| 83 | 60 | copper oxidase [Bacillus] >gb\|EFV71562.1\|CotA | WP_003179495.1 | 65% | 258 | T |
| | | protein [Bacillus sp. BT1B_CT2] >gb\|ADZ57281.1\| | | | | |
| | | laccase [Bacillus sp. LS04] >gb\|EID49890.1\|spore | | | | |
| | | coat protein [Bacillus licheniformis WX-02] | | | | |
| | | >gb\|EQM29388.1\|copper oxidase [Bacillus | | | | |
| | | licheniformis CG-B52] | | | | |
| 84 and 9 | 61 | spore coat protein [Bacillus licheniformis DSM 13 = | YP_077905.1 | 64% | 258 | T |
| | | ATCC 14580] >ref\|YP_006712087.1\|outer spore coat | | | | |
| | | protein CotA [Bacillus licheniformis DSM 13 = ATCC | | | | |
| | | 14580] >ref\|WP_011197606.1\|copper oxidase | | | | |
| | | [Bacillus licheniformis] >gb\|AAU22267.1\|spore coat | | | | |
| | | protein (outer) [Bacillus licheniformis DSM 13 = | | | | |
| | | ATCC 14580] >gb\|AAU39617.1\|outer spore coat | | | | |
| | | protein CotA [Bacillus licheniformis DSM 13 = ATCC | | | | |
| | | 14580] | | | | |
| 85 | 62 | copper oxidase [Bacillus licheniformis S 16] | EWH20929.1 | 64% | 258 | T |
| 86 | 63 | copper oxidase [Oceanobacillus kimchii] | WP_017796468.1 | 61% | 257 | T |
| 87 | 64 | copper oxidase [Bacillus acidiproducens] | WP_018661628.1 | 62% | 261 | S |
| 88 | 65 | hypothetical protein [Bacillus endophyticus] | WP_019395541.1 | 60% | 257 | T |
| 89 | 66 | spore outer coat protein [Oceanobacillus iheyensis | NP_692267.1 | 61% | 257 | T |
| | | HTE831] >ref\|WP_011065752.1\|copper oxidase | | | | |
| | | [Oceanobacillus iheyensis] >dbj\|BAC13302.1\|spore | | | | |
| | | coat protein (outer) [Oceanobacillus iheyensis | | | | |
| | | HTE831] | | | | |
| 90 | 67 | multicopper oxidase type 2 [Bacillus coagulans 36D1] | YP_004860005.1 | 61% | 261 | T |
| | | >ref\|WP_014097300.1\|copper oxidase [Bacillus | | | | |
| | | coagulans] >gb\|AEP01225.1\|multicopper oxidase type | | | | |
| | | 2 [Bacillus coagulans 36D1] | | | | |
| 91 and | 68 | bilirubin oxidase [Bacillus coagulans 2-6] | YP_004569824.1 | 61% | 261 | T |
| 10 | | >ref\|WP_013860324.1\|copper oxidase [Bacillus | | | | |
| | | coagulans] >gb\|AEH54438.1\|Bilirubin oxidase | | | | |
| | | [Bacillus coagulans 2-6] | | | | |
| 92 | 69 | copper oxidase [Bacillus coagulans] | WP_017553860.1 | 61% | 261 | T |
| 93 | 70 | copper oxidase [Bacillus coagulans] | WP_019721501.1 | 60% | 261 | T |

⁽¹⁾Overall identity of selected sequence with SEQ ID NO: 1, the query sequence
⁽²⁾Position number of the selected sequence that corresponds with position 260 in SEQ ID NO: 1.
⁽³⁾Amino acid at a position of the selected sequence that corresponds with position 260 in SEQ ID NO: 1

Analysis of the homologous proteins revealed that all proteins with at least 60% sequence identity to SEQ ID NO: 1 belong to the species of Bacillus. All sequences with at least 60% sequence identity to SEQ ID NO: 1 were copper-dependent oxidases (laccases) and most of them were annotated as spore coat proteins. Thus, it was concluded that sequences with this extent (at least 60%) of identity to SEQ ID NO: 1 represent a highly functionally and structurally related group of proteins that are likely to have similar structural traits and folding pathways.

In other words, the disclosure relates to a spore coat polypeptide with laccase activity wherein the polypeptide comprises an alanine residue at a position corresponding to amino acid 260 of SEQ ID NO: 1. In a preferred embodiment, the polypeptide according to the disclosure is a polypeptide as described above encoded by the genome of a Bacillus species, such as Bacillus subtilis.

None of the 70 laccases from Table 1 (69 sequences from the search plus SEQ ID NO: 1 used as the query sequence) has an alanine residue at a position corresponding to position 260 of SEQ ID NO: 1. Thus, it may be concluded that a laccase with at least 60% sequence identity to SEQ ID NO: 1 comprising an alanine at a position corresponding to position 260 of SEQ ID NO: 1 has not yet been described in the prior art.

It is remarkable that the amino acid corresponding to position 260 in SEQ ID NO: 1 is well conserved within the group of 70 sequences of Table 1. A threonine residue occurs at that position in 68 out of 70 cases (97%) whereas one sequence (SEQ ID NO: 68) appears to have a methionine at that position and one other (SEQ ID NO: 87) has a serine.

It was also observed that the search identified three different groups of sequences. The first group comprises 27 sequences with between 94% and 100% identity with SEQ ID NO: 1. Those sequences were almost all annotated as Bacillus subtilis CotA spore coat proteins, apart from two Bacillus vallismortis CotA (SEQ ID NO: 29 and SEQ ID NO: 49).

Next, there is a second group of 15 sequences with an identity of between 75% and 81% with the sequence of SEQ ID NO: 1.

The third group consisting of 25 members has an identity between 60% and 67% with the sequence of SEQ ID NO: 1. It was found that 67 out of 69 sequences from the search (97%) belonged to either one of these three groups.

Introduction of a specific mutation in a recombinant gene is among the routine skills of a molecular biologist. Specific guidance may be obtained from Methods in Molecular Biology, Vol. 182, “In vitro mutagenesis protocols,” ed. Jeff Braman, Humana Press 2002. There are commercially available kits for performing site-directed mutagenesis (for example, QUIKCHANGE® II XL Site-Directed Mutagenesis kit, Agilent Technologies cat. no. 200521).

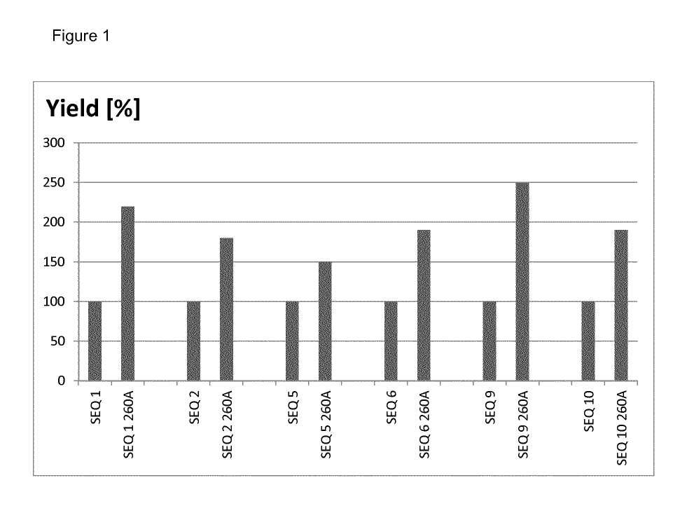

Variants of two representatives of laccases were prepared from each of the above-described three groups. This includes laccases with an amino acid sequence according to SEQ ID NO: 1 and SEQ ID NO: 2 as representatives of group 1 (94% to 100% identity). The sequences of these variants are shown as SEQ ID NO: 3 and SEQ ID NO: 4, respectively, wherein the threonine residue at position 260 of SEQ ID NO: 1 and SEQ ID NO: 2 was replaced by an alanine. When expressed in E. coli, both variants showed an increased yield of active enzyme of 220% and 180%, respectively (FIG. 1). In other words, the volumetric activity of both variants was increased to at least 180%.

As a control experiment, it was determined whether this improved volumetric activity may be attributable to an increased specific activity of the enzyme. This appeared not to be the case. The increase in the amount of mutated enzyme (260A) in the soluble fraction of cell lysate was proportional to the increase in volumetric activity, so it has to be concluded that more variant enzyme may be recovered, thereby completely accounting for the increase in volumetric activity. Hence, the yield of the laccase enzyme is increased rather than its specific activity.

Variants of two representatives of laccases were also prepared from the second group (75% to 81% identity). This concerns laccases with an amino acid sequence according to SEQ ID NO: 5 and SEQ ID NO: 6. The sequences of the variants are shown as SEQ ID NO: 7 and SEQ ID NO: 8, respectively, wherein the amino acid residue at a position corresponding to position 260 of SEQ ID NO: 1 was replaced by an alanine. It should be noted that SEQ ID NO: 5 has a threonine residue at a position corresponding to amino acid 260 of SEQ ID NO: 1, whereas SEQ ID NO: 6 has a methionine residue at that position.

When expressed in E. coli, both variants showed an increased yield of active enzyme of 150% and 190%, respectively. In other words, the volumetric activity of both variants was increased by at least 50% (FIG. 1).

Variants of two representatives of laccases were also prepared from the third group (60% to 67% identity). This concerns laccases with an amino acid sequence according to SEQ ID NO: 9 and SEQ ID NO: 10. The sequences of these variants are shown as SEQ ID NO: 11 and SEQ ID NO: 12, respectively. In SEQ ID NO: 9, amino acid 258 corresponds to amino acid 260 of SEQ ID NO: 1, wherein amino acid 261 of SEQ ID NO: 10 corresponds to amino acid 260 of SEQ ID NO: 1. Both, SEQ ID NO: 9 and SEQ ID NO: 10 have a threonine at the position corresponding to position 260 of SEQ ID NO: 1. That threonine residue was replaced with an alanine in order to arrive at polypeptides with a variant amino acid sequence according to SEQ ID NO: 11 and SEQ ID NO: 12, respectively.

When expressed in E. coli, both variants showed an increased yield of active enzyme of 250% and 190%, respectively (FIG. 1). In other words, the volumetric activity of both variants was increased by at least 90%.

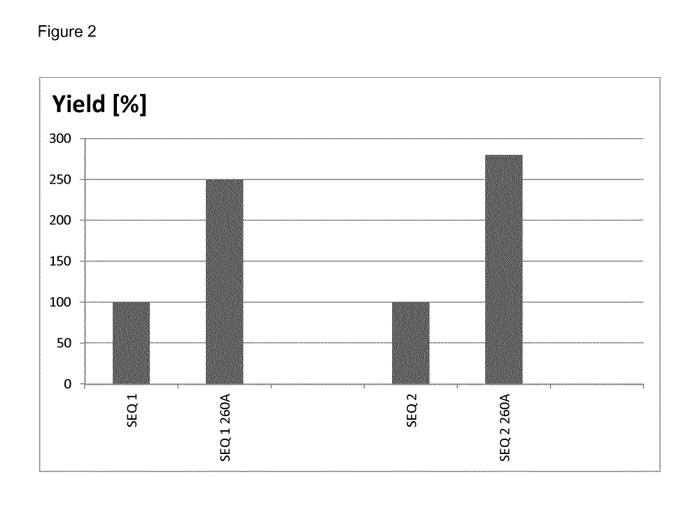

The variants according to SEQ ID NO: 3 and SEQ ID NO: 4 were also expressed in Pichia pastoris. In accordance with the data obtained in a prokaryotic expression system (E. coli, see above) the eukaryotic expression also showed an increased yield. The yield was improved to at least 250% when the expression of the variant sequences was compared with their wild type, SEQ ID NO: 1 and SEQ ID NO: 2, respectively (FIG. 2).

Accordingly, the disclosure relates to a polypeptide with laccase activity comprising an amino acid sequence that is at least 60% identical to the amino acid sequence according to SEQ ID NO: 1, wherein the polypeptide comprises an alanine residue at a position corresponding to position 260 in SEQ ID NO: 1.

This variant amino acid is herein also referred to as amino acid variant 260Ala or 260A. In a preferred embodiment, the polypeptide is isolated.

The above finding that spore coat proteins occur in three distinct groups allows definition of the disclosure in yet another way, such as the structural relationship between the polypeptide according to the disclosure and the reference polypeptides according to the sequences herein. Hence, the disclosure relates to a polypeptide comprising an amino acid sequence that is at least 94% identical to the amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and SEQ ID NO: 12.

The term “at least 94%” is herein used to include at least 95%, such as at least 96%, 97%, 98%, 99% or even 100%. As an example, SEQ ID NO: 1 and SEQ ID NO: 2 are 96% identical, whereas SEQ ID NO: 5 and SEQ ID NO: 6 are 95% identical.

The term “amino acid variant,” “laccase variant,” or “sequence variant” or equivalent has a meaning well recognized in the art and is accordingly used herein to indicate an amino acid sequence that has at least one amino acid difference as compared to another amino acid sequence, such as the amino acid sequence from which it was derived.

The term “at least 60%” is used herein to include at least 61%, such as at least 62%, 63%, 64%, 65%, 66%, 67%, 68%, 69%, 70% or more, such as at least 71%, 72%, 73%, 74%, 75%, 76%, 77%, 78%, 79%, 80% or more, such as at least 81%, 82%, 83%, 84%, 85%, 86%, 87%, 88%, 89%, 90% or more, such as at least 91%, 92%, 93%, 94%, 95%, 96%, 97%, 98%, 99%, or even 100%.

The term “laccase activity” is used herein to mean the capability of a polypeptide to act as a laccase enzyme, which may be expressed as the maximal initial rate of the specific oxidation reaction. Laccase activity may be determined by standard oxidation assays known in the art including, such as, for example, by measurement of oxidation of syringaldazine, according to Sigma online protocol, or according to Cantarella et al. 2003.⁽⁷⁾

An example of determining relative laccase activity is presented in Example 4. Any substrate suitable for the enzyme in question may be used in the activity measurements. A non-limiting example of a substrate suitable for use in assessing the enzymatic activity of laccase variants is ABTS (2,2′-azino-bis(3-ethylbenzothiazoline-6-sulphonic acid). Laccases are able to oxidize this substrate.

As used herein, the term “increased (or improved) laccase-specific activity” refers to a laccase activity higher than that of a corresponding non-mutated laccase enzyme under the same conditions.

The term “increased yield” or equivalent means that the yield of the active enzyme from the same culture volume obtained in a standard purification or recovery protocol is improved by at least 50% or a factor 1.5. The increase may be even more, such as a factor 2, 2.5, 3, 4, 5, 6, 7, 8, 9, 10, 11, 12, 13, 14, 15 or more.

Recovery of a laccase variant produced by a host cell may be performed by any technique known to those skilled in the art. Possible techniques include, but are not limited to, secretion of the protein into the expression medium, and purification of the protein from cellular biomass. The production method may further comprise a step of purifying the laccase variant obtained. For thermostable laccases, non-limiting examples of such methods include heating of the disintegrated cells and removing coagulated thermo-labile proteins from the solution. For secreted proteins, non-limiting examples of such methods include ion exchange chromatography, and ultra-filtration of the expression medium. It is important that the purification method of choice is such that the purified protein retains its activity, preferably its laccase activity.

The laccase variants according to this disclosure may be used in a wide range of different industrial processes and applications, such as cellulose recovery from lignocellulosic biomass, decreasing refining energy in wood refining and pulp preparation, in pulp delignification, textile dye bleaching, wastewater detoxification, xenobiotic detoxification, and detergent manufacturing.

Mutations corresponding to the 260A mutation may be introduced into any of the amino acid sequences disclosed herein, or other homologous sequences, by standard methods known in the art, such as site-directed mutagenesis. In this way, the yield of the laccases from a heterologous expression system may be improved.

Kits for performing site-directed mutagenesis are commercially available in the art (e.g., QUIKCHANGE® II XL Site-Directed Mutagenesis kit by Agilent Technologies). Further suitable methods for introducing the above mutations into a recombinant gene are disclosed, e.g., in Methods in Molecular Biology, 2002.⁽⁸⁾

Thus, some embodiments of this disclosure relate to laccase variants or mutants that comprise Alanine (Ala) in a position that corresponds to the position 260 of the amino acid sequence depicted in SEQ ID NO: 1, and have an increased yield as compared to that of a corresponding non-mutated control when expressed in a heterologous expression system.

The term “heterologous expression system” or equivalent means a system for expressing a DNA sequence from one host organism in a recipient organism from a different species or genus than the host organism. The most prevalent recipients, known as heterologous expression systems, are usually chosen because they are easy to transfer DNA into or because they allow for a simpler assessment of the protein's function. Heterologous expression systems are also preferably used because they allow the upscaling of the production of a protein encoded by the DNA sequence in an industrial process. Preferred recipient organisms for use as heterologous expression systems include bacterial, fungal and yeast organisms, such as, for example, Escherichia coli, Bacillus, Corynebacterium, Pseudomonas, Pichia pastoris, Saccharomyces cerevisiae, Yarrowia lipolytica, filamentus fungi and many more systems well known in the art.

As used herein, the degree of identity between two or more amino acid sequences is equivalent to a function of the number of identical positions shared by the sequences (i.e., % identity=number of identical positions divided by the total number of positions×100), excluding gaps, which need to be introduced for optimal alignment of the two sequences, and overhangs. The comparison of sequences and determination of percent identity between two or more sequences can be accomplished using standard methods known in the art. For example, a freeware conventionally used for this purpose is “Align” tool at NCBI recourse http://blast.ncbi.nlm.nih.gov/Blast.cgi?PAGE_TYPE=BlastSearch&BLAST_SPEC=blast2seq& LINK_LOC=align2seq

The present laccase polypeptides or proteins may be fused to additional sequences, by attaching or inserting, including, but not limited to, affinity tags, facilitating protein purification (S-tag, maltose binding domain, chitin binding domain), domains or sequences assisting folding (such as thioredoxin domain, SUMO protein), sequences affecting protein localization (periplasmic localization signals, etc.), proteins bearing additional function, such as green fluorescent protein (GFP), or sequences representing another enzymatic activity. Other suitable fusion partners for the present laccases are known to those skilled in the art.

This disclosure also relates to polynucleotides encoding any of the laccase variants disclosed herein. Means and methods for cloning and isolating such polynucleotides are well known in the art.

Furthermore, this disclosure relates to a vector comprising a polynucleotide according to the disclosure, optionally operably linked to one or more control sequences. Suitable control sequences are readily available in the art and include, but are not limited to, promoter, leader, polyadenylation, and signal sequences.

Laccase variants according to various embodiments of this disclosure may be obtained by standard recombinant methods known in the art. Briefly, such a method may comprise the steps of i) culturing a desired recombinant host cell under conditions suitable for the production of a present laccase polypeptide variant, and ii) recovering the polypeptide variant obtained. The polypeptide may then optionally be further purified.

A large number of vector-host systems known in the art may be used for recombinant production of laccase variants. Possible vectors include, but are not limited to, plasmids or modified viruses that are maintained in the host cell as autonomous DNA molecule or integrated in genomic DNA. The vector system must be compatible with the host cell used as is well known in the art. Non-limiting examples of suitable host cells include bacteria (e.g., E. coli, bacilli), yeast (e.g., Pichia Pastoris, Saccharomyces Cerevisae), fungi (e.g., filamentous fungi), and insect cells (e.g., Sf9).

A polypeptide according to the disclosure may be advantageously used in an application selected from the group consisting of pulp delignification, degrading or decreasing the structural integrity of lignocellulosic material, textile dye bleaching, wastewater detoxification, xenobiotic detoxification, production of a sugar from a lignocellulosic material and recovering cellulose from a biomass.

In yet other terms, the disclosure relates to a method for improving the yield of a polypeptide with laccase activity in a heterologous expression system comprising the step of altering the amino acid at a position corresponding to position 260 in SEQ ID NO: 1 to an alanine residue.

### EXAMPLES

### Example 1: Construction of Laccases with Improved Properties

Mutations as described herein were introduced into various recombinant genes by standard site-directed mutagenesis essentially as described in WO 2013/038062. In more detail, to introduce mutation T260A into the gene of SEQ ID NO: 1, two separate PCRs were carried out:

(1) with primers Primer1
(SEQ ID NO: 13)
GAAATTAATACGACTCACTATAGG
and
Primer2 (Seq1)
(SEQ ID NO: 14)
GAGGCGTTGATGACGCGAAAGCGGTATTTCCTCGG,
(2) with Primer3 (Seq1)
(SEQ ID NO: 15)
CTTTCGCGTCATCAACGCCTCCAATgCaAGAACC
and
Primer 4
(SEQ ID NO: 16)
GGTTATGCTAGTTATTGCTCAGCGGTG.

In both reactions, recombinant gene without the mutation was used as the template. Primer1 and primer4 bind inside the vector sequence and not specific to the recombinant gene. Primer2 and primer3 bind inside the recombinant gene and their binding sites overlap. Primer3 binding site contains the mutation site. Primer3 represents the mutated (desired) sequence, which is not 100% matching the template (lower case type font in the primer sequence indicate the mis-matched nucleotides); however, the primer has enough affinity and specificity to the binding site to produce the desired PCR product. Purified PCR products from reactions (1) and (2) were combined and used as template for PCR reaction with Primer 1 and Primer 4. The product of this reaction, containing the mutant sequence of the gene, was cloned in a plasmid vector for expression in E. coli.

The same protocol and the same primers were used for introducing the T260A mutation into the gene encoding the polypeptide comprising SEQ ID NO: 2.

Similarly, for introducing a T260A mutation into other genes (corresponding to SEQ ID NO: 5, SEQ ID NO: 6, SEQ ID NO: 9 and SEQ ID NO: 10) the same Primer1 and Primer4 were used, whereas Primer2 and Primer3 were specific for each gene.

In the polypeptide comprising the sequence according to SEQ ID NO: 5, there is a threonine at position 260, the position corresponding to amino acid 260 in SEQ ID NO: 1. For introducing the T260A mutation into the polypeptide comprising the sequence according to SEQ ID NO: 5, the following primer3 and primer2 were used:

Primer3 (seq5)
(SEQ ID NO: 17)
CCGTATCCTTAACGCCTCAAATgCGAGAACATTTTC
Primer2 (seq5)
(SEQ ID NO: 18)
TTTGAGGCGTTAAGGATACGGAAACGATATGTC.

In the polypeptide comprising the sequence according to SEQ ID NO: 6, there is a methionine at position 260, the position corresponding to amino acid 260 in SEQ ID NO: 1. For introducing the M260A mutation into the polypeptide comprising the sequence according to SEQ ID NO: 6, the following primers3 and 2 were used:

Primer3 (seq6)
(SEQ ID NO: 19)
CCGCATCCTTAACGCCTCAAATgcGAGATCATTTA
Primer2 (seq6)
(SEQ ID NO: 20)
ATTTGAGGCGTTAAGGATGCGGAAACGGTATG.

In the polypeptide comprising the sequence according to SEQ ID NO: 9, there is a threonine at position 258, the position corresponding to amino acid 260 in SEQ ID NO: 1. For introducing the T258A mutation into the polypeptide comprising the sequence according to SEQ ID NO: 9, the following primers3 and 2 were used:

Primer3 (seq9)
(SEQ ID NO: 21)
CGTTTTCGGATACTGAACGCCTCCAATgCGAGAATCT
Primer2 (seq9)
(SEQ ID NO: 22)
TGGAGGCGTTCAGTATCCGAAAACGGTATTTTCG.

In the polypeptide comprising the sequence according to SEQ ID NO: 10, there is a threonine at position 261, the position corresponding to amino acid 260 in SEQ ID NO: 1. For introducing the T261A mutation into the polypeptide comprising the sequence according to SEQ ID NO: 10, the following primers3 and 2 were used:

Primer3 (seq10)
(SEQ ID NO: 23)
GGTTCCGGATTGTCAATGCGTCCAACgCGCGGGCCTAT
Primer2 (seq10)
(SEQ ID NO: 24)
TTGGACGCATTGACAATCCGGAACCGGTATTTTCGCGGC

The sequences as described herein and above are shown in Table 2.

TABLE 2
Sequences of SEQ ID NOs: 1-24.

| SEQ | | | |
|---|---|---|---|
| ID | | | |
| NO: | Name | Organism | Sequence |
| 1 | COT1 | B. | MTLEKFVDALPIPDTLKPVQQTTEKTYYEVTMEECAHQLHRDLPPTRLWGYNGLFPGPTIEVKRNEN |
| | | subtilis | VYVKWMNNLPSEHFLPIDHTIHHSDSQHEEPEVKTVVHLHGGVTPDDSDGYPEAWFSKDFEQTGPYF |
| | | | KREVYHYPNQQRGAILWYHDHAMALTRLNVYAGLVGAYIIHDPKEKRLKLPSGEYDVPLLITDRTIN |
| | | | EDGSLFYPSGPENPSPSLPKPSIVPAFCGDTILVNGKVWPYLEVEPRKYRFRVINASNTRTYNLSLD |
| | | | NGGEFIQIGSDGGLLPRSVKLNSFSLAPAERYDIIIDFTAYEGESIILANSEGCGGDANPETDANIM |
| | | | QFRVTKPLAQKDESRKPKYLASYPSVQNERIQNIRTLKLAGTQDEYGRPVLLLNNKRWHDPVTEAPK |
| | | | AGTTEIWSIVNPTQGTHPIHLHLVSFRVLDRRPFDIARYQERGELSYTGPAVPPPPSEKGWKDTIQA |
| | | | HAGEVLRIAVTFGPYSGRYVWHCHILEHEDYDMMRPMDITDPHK |
| 2 | COT2 | B. | MTLEKFVDALPIPDTLKPVQQSKEKTYYEVTMEECTHQLHRDLPPTRLWGYNGLFPGPTIEVKRNEN |
| | | subtilis | VYVKWMNNLPSTHFLPIDHTIHHSDSQHEEPEVKTVVHLHGGVTPDDSDGYPEAWFSKDFEQTGPYF |
| | | | KREVYHYPNQQRGAILWYHDHAMALTRLNVYAGLVGAYIIHDPKEKRLKLPSEEYDVPLLITDRTIN |
| | | | EDGSLFYPSGPENPSPSLPNPSIVPAFCGETILVNGKVWPYLEVEPRKYRFRVINASNTRTYNLSLD |
| | | | NGGEFIQIGSDGGLLPRSVKLTSFSLAPAERYDIIIDFTAYEGQSIILANSAGCGGDVNPETDANIM |
| | | | QFRVTKPLAQKDESRKPKYLASYPSVQNERIQNIRTLKLAGTQDEYGRPVLLLNNKRWHDPVTEAPK |
| | | | AGTTEIWSIINPTRGTHPIHLHLVSFRVIDRRPFDIAHYQESGALSYTGPAVPPPPSEKGWKDTIQA |
| | | | HAGEVLRIAATFGPYSGRYVWHCHILEHEDYDMMRPMDITDPHKSDPNSSSVDKLHRTRAPPPPPLR |
| | | | SGC |
| 3 | 1260A COT1 | B. | MTLEKFVDALPIPDTLKPVQQTTEKTYYEVTMEECAHQLHRDLPPTRLWGYNGLFPGPTIEVKRNEN |
| | | subtilis | VYVKWMNNLPSEHFLPIDHTIHHSDSQHEEPEVKTVVHLHGGVTPDDSDGYPEAWFSKDFEQTGPYF |
| | | | KREVYHYPNQQRGAILWYHDHAMALTRLNVYAGLVGAYIIHDPKEKRLKLPSGEYDVPLLITDRTIN |
| | | | EDGSLFYPSGPENPSPSLPKPSIVPAFCGDTILVNGKVWPYLEVEPRKYRFRVINASNARTYNLSLD |
| | | | NGGEFIQIGSDGGLLPRSVKLNSFSLAPAERYDIIIDFTAYEGESIILANSEGCGGDANPETDANIM |
| | | | QFRVTKPLAQKDESRKPKYLASYPSVQNERIQNIRTLKLAGTQDEYGRPVLLLNNKRWHDPVTEAPK |
| | | | AGTTEIWSIVNPTQGTHPIHLHLVSFRVLDRRPFDIARYQERGELSYTGPAVPPPPSEKGWKDTIQA |
| | | | HAGEVLRIAVTFGPYSGRYVWHCHILEHEDYDMMRPMDITDPHK |
| 4 | T260A COT2 | B. | MTLEKFVDALPIPDTLKPVQQSKEKTYYEVTMEECTHQLHRDLPPTRLWGYNGLFPGPTIEVKRNEN |
| | | subtilis | VYVKWMNNLPSTHFLPIDHTIHHSDSQHEEPEVKTVVHLHGGVTPDDSDGYPEAWFSKDFEQTGPYF |
| | | | KREVYHYPNQQRGAILWYHDHAMALTRLNVYAGLVGAYIIHDPKEKRLKLPSEEYDVPLLITDRTIN |
| | | | EDGSLFYPSGPENPSPSLPNPSIVPAFCGETILVNGKVWPYLEVEPRKYRFRVINASNARTYNLSLD |
| | | | NGGEFIQIGSDGGLLPRSVKLTSFSLAPAERYDIIIDFTAYEGQSIILANSAGCGGDVNPETDANIM |
| | | | QFRVTKPLAQKDESRKPKYLASYPSVQNERIQNIRTLKLAGTQDEYGRPVLLLNNKRWHDPVTEAPK |
| | | | AGTTEIWSIINPTRGTHPIHLHLVSFRVIDRRPFDIAHYQESGALSYTGPAVPPPPSEKGWKDTIQA |
| | | | HAGEVLRIAATFGPYSGRYVWHCHILEHEDYDMMRPMDITDPHKSDPNSSSVDKLHRTRAPPPPPLR |
| | | | SGC |
| 5 | Spore copper | B. | MALEKFADEL PIIETLKPQK TSNGSTYYEV TMKECFHKLH RDLPPTRLWG YNGLFPGPTI |
| | dependent | amylolique- | DVNQDENVYI KWMNDLPDKH FLPVDHTIHH SEGGHQEPDV KTVVHLHGGA TPPDSDGYPE |
| | laccase | faciens | AWFTRDFKEK GPYFEKEVYH YPNKQRGALL WYHDHAMAIT RLNVYAGLAG MYIIRERKEK |
| | | | QLKLPAGEYD VPLMIMDRTL NDDGSLFYPS GPDNPSETLP NPSIVPFLCG NTILVNGKAW |
| | | | PYMEVEPRTY RFRILNASNT RTFSLSLNNG GRFIQIGSDG GLLPRSVKTQ SISLAPAERY |
| | | | DVLIDFSAFD GEHIILTNGT GCGGDVNPDT DANVMQFRVT KPLKGEDTSR KPKYLSAMPD |
| | | | MTSKRIHNIR TLKLTNTQDK YGRPVLTLNN KRWHDPVTEA PRLGSTEIWS IINPTRGTHP |
| | | | IHLHLVSFQV LDRRPFDLER YNKFGDIVYT GPAVPPPPSE KGWKDTVQAH SGEVIRIAAT |
| | | | FAPYSGRYVW HCHILEHEDY DMMRPMDVTE KQ |
| 6 | copper | B. | MALEKFADEL PIIETLKPQK KSDGSTYYEV TMKECFHKLH RDLPPTRLWG YNGLFPGPTI |
| | oxidase | siamensis | DVNQGESIYV KWMNDLPDKH FLPVDHTIHH SESGHQEPDV RTVVHLHGGE TPPDSDGYPE |
| | | | AWFTRDFKET GPYFEKEVYH YPNKQRGALL WYHDHAMAAT RLNVYAGLAG MYIIRERKEK |
| | | | QLKLPAGEYD VPLMILDRTL NDDGSLSYPS GPDNPSETLP TPSIVPFLCG NTILVNGKAW |
| | | | PYMEVEPRTY RFRILNASNM RSFTLSLNNG GRFIQIGSDG GLLPRSVRTQ TISLAPAERY |
| | | | DVLIDFSAFD GEHIILTNGT GCGGDVDPDT DANVMQFRVT KPLKGEDTSR KPKYLSAMPD |
| | | | MTSKRIHNIR TLKLTNTQDK YGRPVLTLNN KRWHDPVTEA PKLGTTEIWS IINPMGGTHP |
| | | | IHLHLVSFQV LDRRPFDLER YNKFGDIVYT GPAVPPPPSE KGWKDTVQAH SGEVIRIAAT |
| | | | FAPYSGRYVW HCHILEHEDY DMMRPMDVTD KQ |
| 7 | T260A Spore | B. | MALEKFADEL PIIETLKPQK TSNGSTYYEV TMKECFHKLH RDLPPTRLWG YNGLFPGPTI |
| | copper- | amylolique- | DVNQDENVYI KWMNDLPDKH FLPVDHTIHH SEGGHQEPDV KTVVHLHGGA TPPDSDGYPE |
| | dependent | faciens | AWFTRDFKEK GPYFEKEVYH YPNKQRGALL WYHDHAMAIT RLNVYAGLAG MYIIRERKEK |
| | laccase | | QLKLPAGEYD VPLMIMDRTL NDDGSLFYPS GPDNPSETLP NPSIVPFLCG NTILVNGKAW |
| | | | PYMEVEPRTY RFRILNASNA RTFSLSLNNG GRFIQIGSDG GLLPRSVKTQ SISLAPAERY |
| | | | DVLIDFSAFD GEHIILTNGT GCGGDVNPDT DANVMQFRVT KPLKGEDTSR KPKYLSAMPD |
| | | | MTSKRIHNIR TLKLTNTQDK YGRPVLTLNN KRWHDPVTEA PRLGSTEIWS IINPTRGTHP |
| | | | IHLHLVSFQV LDRRPFDLER YNKFGDIVYT GPAVPPPPSE KGWKDTVQAH SGEVIRIAAT |
| | | | FAPYSGRYVW HCHILEHEDY DMMRPMDVTE KQ |
| 8 | M260A copper | B. | MALEKFADEL PIIETLKPQK KSDGSTYYEV TMKECFHKLH RDLPPTRLWG YNGLFPGPTI |
| | oxidase | siamensis | DVNQGESIYV KWMNDLPDKH FLPVDHTIHH SESGHQEPDV RTVVHLHGGE TPPDSDGYPE |
| | | | AWFTRDFKET GPYFEKEVYH YPNKQRGALL WYHDHAMAAT RLNVYAGLAG MYIIRERKEK |
| | | | QLKLPAGEYD VPLMILDRTL NDDGSLSYPS GPDNPSETLP TPSIVPFLCG NTILVNGKAW |
| | | | PYMEVEPRTY RFRILNASNA RSFTLSLNNG GRFIQIGSDG GLLPRSVRTQ TISLAPAERY |
| | | | DVLIDFSAFD GEHIILTNGT GCGGDVDPDT DANVMQFRVT KPLKGEDTSR KPKYLSAMPD |
| | | | MTSKRIHNIR TLKLTNTQDK YGRPVLTLNN KRWHDPVTEA PKLGTTEIWS IINPMGGTHP |
| | | | IHLHLVSFQV LDRRPFDLER YNKFGDIVYT GPAVPPPPSE KGWKDTVQAH SGEVIRIAAT |
| | | | FAPYSGRYVW HCHILEHEDY DMMRPMDVTD KQ |
| 9 | Spore coat | B. | MKLEKFVDRLPIPQVLQPQSKSKEMTYYEVTMKEFQQQLHRDLPPTRLFGYNGVYPGPTFEVQKHEK |
| | protein | licheniformis | VAVKWLNKLPDRHFLPVDHTLHDDGHHEHEVKTVVHLHGGCTPADSDGYPEAWYTKDFHAKGPFFER |
| | | | EVYEYPNEQDATALWYHDHAMAITRLNVYAGLVGLYFIRDREERSLNLPKGEYEIPLLIQDKSFHED |
| | | | GSLFYPRQPDNPSPDLPDPSIVPAFCGDTILVNGKVWPFAELEPRKYRFRILNASNTRIFELYFDHD |
| | | | ITCHQIGTDGGLLQHPVKVNELVIAPAERCDIIVDFSRAEGKTVTLKKRIGCGGQDADPDTDADIMQ |
| | | | FRISKPLKQKDTSSLPRILRKRPFYRRHKINALRNLSLGAAVDQYGRPVLLLNNTKWHEPVTETPAL |
| | | | GSTEIWSIINAGRAIHPIHLHLVQFMILDHRPFDIERYQENGELVFTGPAVPPAPNEKGLKDTVKVP |
| | | | PGSVTRIIATFAPYSGRYVWHCHILEHEDYDMMRPLEVTDVRHQ |
| 10 | Laccase | B. | MSPNLEKFVDRLPLAEKIRPVREEGGIAYYEVTMEEFRQKLHRDLRPTRLWGYNRRFPGPLFDVPHG |
| | | coagulans | KKIRVKWTNHLPQRHFLPIDPTILDGMGTDFPEVRTVVHLHGGETKPDSDGYPEAWFTRDFNETGPA |
| | | | FKNEVYEYSNKQRPATLWYHDHAIGITRLNVYAGLAGMYIIRDQKEKVFHLPSGKYEIPLLLTDRTF |
| | | | NNDGSLFYPRQPQNPGPGTPDPSVVPFFLGDTILVNGKVWPYLEVEPRKYRFRIVNASNTRAYQLYL |
| | | | DSGQAFYQIGTDGGLLRRPVQVGNLALEPAERADLILDFSEYAGQTILLKNDLGPNADPADQTGDVM |
| | | | QFRVVLPVSGEDTSRIPPSLSSIPVPSSQNVSAIRHLKLTGATDSYGRPLLLLDKKRWMDPVTEMPR |
| | | | LGTTEIWSLANTTAFTHPIHIHLVQFQILDRRPFDLDLYNETGQIVYTGPATPPEPSERGFKDTVAA |
| | | | PGGQITRVMMRFSPYAGDYVWHCHILEHEDYDMMRPFQVIDPDLPESDSPLSD |
| 11 | T260A Spore | B. | MKLEKFVDRLPIPQVLQPQSKSKEMTYYEVTMKEFQQQLHRDLPPTRLFGYNGVYPGPTFEVQKHEK |
| | coat protein | licheniformis | VAVKWLNKLPDRHFLPVDHTLHDDGHHEHEVKTVVHLHGGCTPADSDGYPEAWYTKDFHAKGPFFER |
| | | | EVYEYPNEQDATALWYHDHAMAITRLNVYAGLVGLYFIRDREERSLNLPKGEYEIPLLIQDKSFHED |
| | | | GSLFYPRQPDNPSPDLPDPSIVPAFCGDTILVNGKVWPFAELEPRKYRFRILNASNARIFELYFDHD |
| | | | ITCHQIGTDGGLLQHPVKVNELVIAPAERCDIIVDFSRAEGKTVTLKKRIGCGGQDADPDTDADIMQ |
| | | | FRISKPLKQKDTSSLPRILRKRPFYRRHKINALRNLSLGAAVDQYGRPVLLLNNTKWHEPVTETPAL |
| | | | GSTEIWSIINAGRAIHPIHLHLVQFMILDHRPFDIERYQENGELVFTGPAVPPAPNEKGLKDTVKVP |
| | | | PGSVTRIIATFAPYSGRYVWHCHILEHEDYDMMRPLEVTDVRHQ |
| 12 | T260A | B. | MSPNLEKFVDRLPLAEKIRPVREEGGIAYYEVTMEEFRQKLHRDLRPTRLWGYNRRFPGPLEDVPHG |
| | Laccase | coagulans | KKIRVKWTNHLPQRHFLPIDPTILDGMGTDEPEVRTVVHLHGGETKPDSDGYPEAWFTRDFNETGPA |
| | | | FKNEVYEYSNKQRPATLWYHDHAIGITRLNVYAGLAGMYIIRDQKEKVFHLPSGKYEIPLLLTDRTF |
| | | | NNDGSLFYPRQPQNPGPGTPDPSVVPFFLGDTILVNGKVWPYLEVEPRKYRFRIVNASNARAYQLYL |
| | | | DSGQAFYQIGTDGGLLRRPVQVGNLALEPAERADLILDFSEYAGQTILLKNDLGPNADPADQTGDVM |
| | | | QFRVVLPVSGEDTSRIPPSLSSIPVPSSQNVSAIRHLKLTGATDSYGRPLLLLDKKRWMDPVTEMPR |
| | | | LGTTEIWSLANTTAFTHPIHIHLVQFQILDRRPFDLDLYNETGQIVYTGPATPPEPSERGFKDTVAA |
| | | | PGGQITRVMMRFSPYAGDYVWHCHILEHEDYDMMRPFQVIDPDLPESDSPLSD |
| 13 | primer 1 | B. spec | GAAATTAATACGACTCACTATAGG |
| 14 | primer 2 seq1 | B. spec | GAGGCGTTGATGACGCGAAAGCGGTATTTCCTCGG |
| 15 | primer 3 seq1 | B. spec | CTTTCGCGTCATCAACGCCTCCAATgCaAGAACC |
| 16 | primer 4 | B. spec | GGTTATGCTAGTTATTGCTCAGCGGTG |
| 17 | primer 3 seq5 | B. spec | CCGTATCCTTAACGCCTCAAATgCGAGAACATTTTC |
| 18 | primer 2 seq5 | B. spec | TTTGAGGCGTTAAGGATACGGAAACGATATGTC |
| 19 | primer 3 seq6 | B. spec | CCGCATCCTTAACGCCTCAAATgcGAGATCATTTA |
| 20 | primer 2 seq6 | B. spec | ATTTGAGGCGTTAAGGATGCGGAAACGGTATG |
| 21 | primer 3 seq9 | B. spec | cgttttcggatactgaacgcctccaatGcgagaatct |
| 22 | primer 2 seq9 | B. spec | tggaggcgttcagtatccgaaaacggtattttcg |
| 23 | primer 3 seq10 | B. spec | ggttccggattgtcaatgcgtccaacGcgcgggcctat |
| 24 | primer 2 seq10 | B. spec | ttggacgcattgacaatccggaaccggtattttcgcggc |

### Example 2: Heterologous Expression of Variant and Non-Mutated Laccases

Variant laccases were expressed in E. coli and Pichia pastoris.

For expression in Pichia Pastoris, recombinant genes were cloned into a commercial Pichia Pastoris expression vector pPICZ-A available from Invitrogen (Life Technologies). This vector provides secreted protein expression under the control of methanol inducible AOX1 promoter upon integration of the construct into genomic DNA of the yeast cell.

Linearized plasmid DNA was introduced into yeast cells by electroporation, and clones with integrated recombinant gene were selected on agar medium plates with Zeocin (25 ug/ml). Ten colonies from each construct were tested in small liquid cultures (3 ml) with 72-hour cultivation in humidified shaker at 28° C. according to the plasmid manufacturer manual (http://tools.lifetechnologies.com/content/sfs/manuals/ppiczalpha_man.pdf). The medium recommended by the manufacturer was supplemented with 1 mM CuCl, as laccase protein contains copper as a cofactor. Activity in the medium was measured by ABTS oxidation (see Example 4), and the two best producing clones were selected for each gene. Parallel cultures of the selected clones were gown in flask scale according to the plasmid manufacturer manual (see above) at 28° C. for 105 hours. Cells were removed by centrifugation and medium containing the recombinant protein was collected. These preparations were used for comparison of volumetric activities of variant and non-mutated genes.

For recombinant expression in E. coli, recombinant genes were cloned into pET-28 commercial expression vector under the control of T7 bacteriophage promoter. Protein production was carried out in E. coli BL21(DE3) strain according to the plasmid manufacturer protocol http://richsingiser.com/4402/Novagen%20pET%20system%20manual.pdf. The medium recommended by the manufacturer was supplemented with 1 mM CuCl, as laccase protein contains copper as a cofactor. The incubation temperature for protein production was 30° C., which was found optimal for maximum yield of the active protein. Cells were lysed using lysis buffer (50 mM Tris-HCl pH 7.4, 1% TRITON® X-100, 1 mM CuCl) and heated at 70° C. for 20 minutes. Coagulated cell debris was removed by centrifugation. The recombinant laccase, being a thermostable protein, remained in soluble fraction. Enzymatic activity was detectable only in soluble fraction. Analysis of soluble and insoluble fractions by gel-electrophoresis reveals that over 90% of the recombinant protein is present in insoluble inactive form as inclusion bodies (in accordance with literature data).

### Example 3: Measurement of Yield

The relative yields of mutated and non-mutated soluble laccases were determined by densitometry of protein bands after denaturing polyacrylamide gel electrophoresis. To this end, samples of soluble proteins after thermal treatment (see Example 2) obtained from parallel cultures of mutated and non-mutated clones, were analyzed by gel-electrophoresis under denaturing conditions (a standard method well known in the art of molecular biology). After staining the gel with Coomassie Brilliant Blue, the gel was scanned to obtain a bitmap image, and intensity of the band corresponding to recombinant laccase was quantified by ImageJ software (a public freeware developed at the National Institute of Health and online available at http://imagej.nih.gov/ij/).

### Example 4: Relative Activity Measurement of Laccase

As stated above, the term “laccase activity” is used herein to mean the capability to act as a laccase enzyme, which may be expressed as the maximal initial rate of the specific oxidation reaction. Relative activity was measured by oxidation of ABTS (2,2′-azino-bis(3-ethylbenzothiazoline-6-sulphonic acid). Reaction course was monitored by change in absorbance at 405 nM (green color development). The appropriate reaction time was determined to provide initial rates of oxidation when color development is linear with time. Substrate (ABTS) concentration was 5 mM to provide maximum initial rates (substrate saturation conditions).

Typically, reactions were carried out in 96-well flat bottom plates, each well contained 2 μl of enzyme preparation in 200 μl of 100 mM Succinic acid pH 5, the reaction was initiated by simultaneous addition of the substrate (22 μl of 50 mM ABTS) in each well. After the reaction time has elapsed, absorbance at 405 nm of the reaction mixtures was determined by a plate reader (Multiscan Go, Thermo Scientific). In order to determine relative activity of mutated laccase, the absorbance of the reference laccase sample was taken for 100%, and relative activity was determined as fraction of this absorbance.

### Example 5: Alignment of Fragments from SEQ ID NO:s 25-93

In order to identify the position corresponding to amino acid 260 of SEQ ID NO: 1, the sequences according to SEQ ID NO: 25-93 were aligned using the standard protein BLAST software as disclosed herein. Fragments of 61 amino acids long from SEQ ID NO:s 25-93, aligned to the corresponding sequence of SEQ ID NO: 1, are shown in Table 3. The amino acid corresponding to amino acid 260T in SEQ ID NO: 1 is underlined in all sequences shown in Table 3.

TABLE 3
Alignment of fragments of SEQ ID NO: 25-93, comparison with SEQ ID NO: 1.

| | | | | Amino Acid |
|---|---|---|---|---|
| Seq | | | | corresponding |
| ID | Fragment of | First | | to position |
| NO: | SEQ ID NO: | aa No | Amino acid sequence alignment | 260T |
| 94 | 1 | 232 | TILVNGKVWPYLEVEPRKYRFRVINASNTRTYNLSLDNGGEFIQIGSDGGLLPRSVKLNSF | 260T |
| 95 | 25 | 232 | TILVNGKVWPYLEVEPRKYRFRVINASNTRTYNLSLDNGGEFIQIGSDGGLLPRSVKLNSF | 260T |
| 96 | 26 | 232 | TILVNGKVWPYLEVEPRKYRFRVINASNTRTYNLSLDNGGEFIQIGSDGGLLPRSVKLNSF | 260T |
| 97 | 27 | 232 | TILVNGKVWPYLEVEPRKYRFRVINASNTRTYNLSLDNGGEFIQIGSDGGLLPRSVKLNSF | 260T |
| 98 | 28 | 232 | TILVNGKAWPYFEVEPRKYRFRVINASNTRTYNLSLDNGGAFIQIGSDGGLLPRSVKLNSF | 260T |
| 99 | 29 | 232 | TILVNGKVWPYLEVEPRKYRFRVINASNTRTYNLSLDNGGEFIQVGSDGGLLPRSVKLNSF | 260T |
| 100 | 30 | 234 | TILVNGKVWPYLEVEPRKYRFRVINASNTRTYNLSLDNGGEFIQIGSDGGLLPRSVKLNSF | 262T |
| 101 | 31 | 232 | TILVNGKVWPYLEVEPRKYRFRVINASNTRTYNLSLDNGGEFIQIGSDGGLLPRSVKLNSF | 260T |
| 102 | 32 | 232 | TILVNGKVWPYLEVEPRKYRFRVINASNTRTYNLSLDNGGEFIQVGSDGGLLPRSVKLNSF | 260T |
| 103 | 33 | 232 | TILVNGKVWPYLEVEPRKYRFRVINASNTRTYNLSLDNGGEFIQIGSDGGLLPRSVKLNSF | 260T |
| 104 | 34 | 233 | TILVNGKVWPYLEVEPRKYRFRVINASNTRTYNLSLDNGGEFIQIGSDGGLLPRSVKLNSF | 262T |
| 105 | 35 | 233 | TILVNGKVWPYLEVEPRKYRFRVINASNTRTYNLSLDNGGDFIQIGSDGGLLPRSVKLNSF | 262T |
| 106 | 36 | 232 | TILVNGKVWPYLEVEPRKYRFRVINASNTRTYNLSLDNGGDFIQIGSDGGLLPRSVKLNSF | 260T |
| 107 | 37 | 232 | TILVNGKVWPYLEVEPRKYRFRVINASNTRTYNLSLDNGGEFIQIGADGGLLPRSVKLNSF | 260T |
| 108 | 38 | 232 | TILVNGKVWPYLEVEPRKYRFRVINASNTRTYNLSLDNGGDFIQIGSDGGLLPRSVKLNSF | 260T |
| 109 | 39 | 232 | TILVNGKVWPYLEVEPRKYRFRVINASNTRTYNLSLDNGGDFIQIGSDGGLLPRSVKLNSF | 260T |
| 110 | 40 | 232 | TILVNGKVWPYLEVEPRKYRFRVINASNTRTYNLSLDNGGDFIQIGSDGGLLPRSVKLNSF | 260T |
| 111 | 41 | 232 | TILVNGKVWPYLEVEPRKYRFRVINASNTRTYNLSLDNGGEFIQIGSDGGLLPRSVKLNSF | 260T |
| 112 | 42 | 232 | TILVNGKAWPYFEVEPRKYRFRVINASNTRTYNLSLDNGGAFIQIGSDGGLLPRSVKLNSF | 260T |
| 113 | 43 | 232 | TILVNGKVWPYLEVEPRKYRFRVINASNTRTYNLSLDNGGDFIQIGSDGGLLPRSVKLNSF | 260T |
| 114 | 44 | 232 | TILVNGKVWPYLEVEPRKYRFRVINASNTRTYNLSLDNGGDFIQIGSDGGLLPRSVKLNSF | 260T |
| 115 | 45 | 232 | TILVNGKVWPYLEVEPRKYRFRVINASNTRTYNLSLDNGGDFIQIGSDGGLLPRSVKLNSF | 260T |
| 116 | 46 | 232 | TILVNGKVWPYLEVEPRKYRFRVINASNTRTYNLSLDNGGDFIQIGSDGGLLPRSVKLNSF | 260T |
| 117 | 47 | 232 | TILVNGKVWPYLEVEPRKYRFRVINASNTRTYNLSLDNGGDFIQIGSDGGLLPRSVKLNSF | 260T |
| 118 | 48 | 232 | TILVNGKVWPYLEVEPRKYRFRVINASNTRTYNLSLDNGGDFIQIGSDGGLLPRSVKLNSF | 260T |
| 119 | 49 | 232 | TILVNGKAWPYLEVEPRKYRFRVINASNTRTYNLSLDNDGEFIQIGSDGGLLPRSVKLNSF | 260T |
| 120 | 50 | 232 | TILVNGKAWPYMEVEPRKYRFRVINASNTRTYNLSLDNGGEFIQIGSDGGLLPRSVKLNSF | 260T |
| 121 | 51 | 232 | TILVNGKAWPYMEVEPRKYRFRVINASNTRTYNLSLDNGGEFIQIGSDGGLLPRSVKLNSF | 260T |
| 122 | 52 | 232 | TILVNGKVWPYLEVEPRKYRFRVINASNTRTYNLSLDNGGDFIQIGSDGGLLPRSVKLNSF | 260T |
| 123 | 53 | 232 | TILVNGKVWPYLEVEPRKYRFRVINASNTRTYNLSLDNGGDFIQIGSDGGLLPRSVKLNSF | 260T |
| 124 | 54 | 232 | TILVNGKAWPYMEVEPRAYRFRIVNASNTRTYNLSLDNGGEFLQVGSDGGLLPRSVKLSSI | 260T |
| 125 | 55 | 232 | TILVNGKAWPYMEVEPRAYRFRIVNASNTRTYNLSLDNGGEFLQVGSDGGLLPRSVKLSSI | 260T |
| 126 | 56 | 232 | TILVNGKAWPYMEVEPRTYRFRILNASNTRTFSLSLNNGGKFIQIGSDGGLLPRSVKTQSI | 260T |
| 127 | 57 | 232 | TILVNGKAWPYMEVEPRTYRFRILNASNTRTFSLSLNNGGKFIQIGSDGGLLPRSVKTQSI | 260T |
| 128 | 58 | 232 | TILVNGKAWPYMEVEPRTYRFRILNASNTRTFSLSLNNGGKFIQIGSDGGLLPRSVKTQSI | 260T |
| 129 | 59 | 232 | TILVNGKAWPYMEVEPRTYRFRILNASNTRTFSLSLNNGGKFIQIGSDGGLLPRSVKTQSI | 260T |
| 130 | 60 | 232 | TILVNGKAWPYMEVEPRTYRFRILNASNTRTFSLSLNNGGKFIQIGSDGGLLPRSVKTQSI | 260T |
| 131 | 61 | 232 | TILVNGKAWPYMEVEPRTYRFRILNASNTRTFSLSLNNGGKFIQIGSDGGLLPRSVKTQSI | 260T |
| 132 | 62 | 232 | TILVNGKAWPYMEVEPRTYRFRILNASNTRTFSLSLNNGGKFIQIGSDGGLLPRSVKTQSI | 260T |
| 133 | 63 | 232 | TILVNGKAWPYMEVEPRTYRFRILNASNTRTFSLSLNNGGKFIQIGSDGGLLPRSVKTQSI | 260T |
| 134 | 64 | 232 | TILVNGKAWPYMEVEPRTYRFRILNASNTRTFSLSLNNGGKFIQIGSDGGLLPRSVKTQSI | 260T |
| 135 | 65 | 232 | TILVNGKAWPYMEVEPRTYRFRILNASNTRTFSLSLNNGGKFIQIGSDGGLLPRSVKTQSI | 260T |
| 136 | 66 | 232 | TILVNGKAWPYMEVEPRTYRFRILNASNTRTFSLSLNNGGKFIQIGSDGGLLPRSVKTQSI | 260T |
| 137 | 67 | 232 | TILVNGKAWPYMEVEPRTYRFRILNASNTRTFSLSLNNGGRFIQIGSDGGLLPRSVKTQSI | 260T |
| 138 | 68 | 232 | TILVNGKAWPYMEVEPRTYRFRILNASNMRSFTLSLNNGGRFIQIGSDGGLLPRSVRTQTI | 260M |
| 139 | 69 | 230 | TILVNGKVWPYAEIEPRKYRFRVLNASNTRIYELYFDSGHAFYQIGTDGGLLQRPAKVESL | 258T |
| 140 | 70 | 232 | TILVNGKVWPYLEVEPRKYRFRILNASNTRTYELHLDNDATILQIGSDGGFLPRPVHHQSF | 260T |
| 141 | 71 | 232 | TILVNGKVWPYLEVEPRKYRFRILNASNTRTYELHLDNDATILQIGSDGGFLPRPVHHQSF | 260T |
| 142 | 72 | 232 | TILVNGKVWPYLEVEPRKYRFRILNASNTRTYELHLDNDATILQIGSDGGFLPRPVHHQSF | 260T |
| 143 | 73 | 232 | TILVNGKVWPYLEVEPRKYRFRILNASNTRTYELHLDNDATILQIGSDGGFLPRPVHHQSF | 260T |
| 144 | 74 | 232 | TILVNGKVWPYLEVEPRKYRFRILNASNTRTYELHLDNDATILQIGSDGGFLPRPVHHQSF | 260T |
| 145 | 75 | 232 | TILVNGKVWPYLEVEPRKYRFRILNASNTRTYELHLDNDATILQIGSDGGFLPRPVQHQSF | 260T |
| 146 | 76 | 232 | TILVNGKVWPYLEVEPRKYRFRILNASNTRTYELHLDNDATILQIGSDGGFLPRPVHHQSF | 260T |
| 147 | 77 | 232 | TILVNGKVWPYLEVEPRKYRFRILNASNTRTYELHLDNDATIMQIGSDGGFLPRPVRHQSF | 260T |
| 148 | 78 | 232 | TILVNGKVWPYLEVEPRKYRFRILNASNTRTYELHLDNDATILQIGSDGGFLPRPVHHQSF | 260T |
| 149 | 79 | 232 | TILVNGKVWPYLEVEPRKYRFRILNASNTRTYELHLDNDATILQIGSDGGFLPRPVHHQSF | 260T |
| 150 | 80 | 230 | TILVNGKVWPYDELEPRKYRFRILNASNTRIFELYFDHDITFHQIGTDGGLLQHPVKVNEL | 258T |
| 151 | 81 | 230 | TILVNGKVWPYDELEPRKYRFRILNASNTRIFELYFDHDITFHQIGTDGGLLQHPVKVNEL | 258T |
| 152 | 82 | 233 | TILVNGKVWPYLEVEPRKYRFRILNASNTRTYELHLDNDATILQIGSDGGFLPRPVHHQSF | 261T |
| 153 | 83 | 230 | TILVNGKVWPFAELEPRKYRFRILNASNTRIFELYFDHDITCHQIGTDGGLLQHPVKVNEL | 258T |
| 154 | 84 | 230 | TILVNGKVWPFAELEPRKYRFRILNASNTRIFELYFDHDITCHQIGTDGGLLQHPVKVNEL | 258T |
| 155 | 85 | 230 | TILVNGKVWPFAEFEPRKYRFRILNASNTRIFELYFDHDITCHQIGTDGGLLQHPVKVNEL | 258T |
| 156 | 86 | 229 | AILVNGKAWPYIDVEPRKYRFRLLNASNTRTYRLSMNEELPIYQIGSDGGLLRKSIPTRQI | 257T |
| 157 | 87 | 233 | TILVNGKIWPYLEVEPRKYRFRVIDVSNSRPYQLYLDSGQPLYQIGTDGGLLRRPVKLERL | 261S |
| 158 | 88 | 229 | TILVNGKVWPYLEVEPRKYRFRLLNASNTRAYQLYLDSGQSFHQIGSDGGLLQKSVHLKKF | 257T |
| 159 | 89 | 229 | TILVNGKAWPYMDVEPRKYRFRLVNASNTRTYRISLNNDVPIYQIGSDGGLLRKSIPTRQF | 257T |
| 160 | 90 | 233 | TILVNGKVWPYLEVEPRKYRFRIVNASNTRAYRLYLDSGQAFYQIGTDGGLLRRPVQVENL | 261T |
| 161 | 91 | 233 | TILVNGKVWPYLEVEPRKYRFRIVNASNTRAYQLYLDSGQAFYQIGTDGGLLRRPVQVGNL | 261T |
| 162 | 92 | 233 | TILVNGKVWPYLEVEPRKYRFRIVNASNTRAYQLYLDSGQAFYQIGTDGGLLRRPVQVGNL | 261T |
| 163 | 93 | 233 | TILVNGKVWPYLEVEPRKYRFRIVNASNTRAYQLYLDSGQAFYQIGTDGGLLRRPVQVGNL | 261T |

### REFERENCES

1. Martins L. O., C. M. Soares, M. M. Pereira, M. Teixeira, T. Costa, and G. H. Jones, et al. Molecular and biochemical characterization of a highly stable bacterial laccase that occurs as a structural component of the Bacillus subtilis endospore coat. J. Biol. Chem. 2002; 277:18849-59.
2. Bento I., L. O. Martins, G. Gato Lopes, M. Arménia Carrondo, and P. F. Lindley. Dioxygen reduction by multi-copper oxidases; a structural perspective. Dalton Trans. 2005; 21:3507-13.
3. Brissos V., L. Pereira, F. D. Munteanu, A. Cavaco-Paulo, and L. O. Martins. Expression system of CotA-laccase for directed evolution and high-throughput screenings for the oxidation of high-redox potential dyes. Biotechnol. J. 2009; 4:558-63.
4. Suzuki T., K. Endo, M. Ito, H. Tsujibo, K. Miyamoto, and Y. Inamori. A thermostable laccase from Streptomyces lavendulae REN-7: purification, characterization, nucleotide sequence and expression. Biosci. Biotechnol. Biochem. 2003; 67:2167-75.
5. Kumar et al., “Combined sequence and structure analysis of the fungal laccase family,” Biotechnol. Bioeng. 83:386-394, 2003;
6. Morozova et al., “Blue laccases,” Biochemistry (Moscow) 72:1136-1150 (2007).
7. Cantarella et al., Determination of laccase activity in mixed solvents: Comparison between two chromogens in a spectrophotometric assay,” Biotechnology and Bioengineering V. 82 (4):395-398 (2003).
8. Methods in Molecular Biology, Vol 182, “In vitro mutagenesis protocols,” ed. Jeff Braman, Humana Press (2002).

1. A polypeptide with laccase activity, the polypeptide comprising:
at least 60% sequence identity to the amino acid sequence according to SEQ ID NO: 1, and
an alanine residue at a position corresponding to amino acid 260 of SEQ ID NO: 1.

2. The polypeptide of claim 1, wherein the polypeptide is a spore coat protein.

3. The polypeptide of claim 1, wherein the polypeptide is encoded by the genome of a Bacillus species.

4. The polypeptide of claim wherein the Bacillus species is Bacillus subtilis.

5. The polypeptide of claim 1, wherein the polypeptide comprises at least 94% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12.

6. The polypeptide of claim 1, wherein the polypeptide is an isolated polypeptide.

7. A composition comprising the polypeptide of claim 1.

8. A nucleic acid molecule encoding the polypeptide of claim 1.

9. A vector comprising the nucleic acid molecule of claim 8.

10. A composition comprising the nucleic acid molecule of claim 8.

11. A recombinant host cell comprising the nucleic acid molecule of claim 8.

12. The recombinant host cell according to claim 11, wherein the host cell is selected from the group consisting of Escherichia coli, Bacillus, Corynebacterium, Pseudomonas, Pichia pastoris, Saccharomyces cerevisiae, Yarrowia lipolytica, filamentous fungi, yeast and insect cells.

13. A method of producing a polypeptide, the method comprising:
culturing the recombinant host cell of claim 11 under conditions suitable for the production of the polypeptide, and
recovering the polypeptide.

14. A method of utilizing the polypeptide of claim 1, the method comprising:
utilizing the polypeptide in an application selected from the group consisting of pulp delignification, degrading or decreasing the structural integrity of lignocellulosic material, textile dye bleaching, wastewater detoxification, xenobiotic detoxification, production of a sugar from a lignocellulosic material, and recovering cellulose from a biomass.

15. A method for improving the yield of a polypeptide with laccase activity in a heterologous expression system, the method comprising:
altering an amino acid at a position corresponding to position 260 in SEQ ID NO: 1 to an alanine residue.

16. A composition comprising the vector of claim 9.

17. A recombinant host cell comprising the vector of claim 9.

18. A recombinant host cell comprising the composition of claim 10.

19. The polypeptide of claim 2, wherein the polypeptide is encoded by the genome of a Bacillus species.

20. The method according to claim 13, further comprising purifying the polypeptide.
